(19) United States
(12) Patent Application Publication
(10) Pub. No.: US 2016/0219829 A1
(43) Pub. Date: Aug. 4, 2016

(54) ANTI-ODOR MATERIAL FOR ANIMAL LITTERS USING PHOSPHOROTRIAMIDES IN POWDER FORM

(71) Applicant: 7905122 CANADA INC., Boucherville (CA)

(72) Inventor: Paul JOLLEZ, Sherbrooke (CA)

(21) Appl. No.: 14/916,812

(22) PCT Filed: Sep. 5, 2014
(86) PCT No.: PCT/CA2014/050844

§ 371 Date: Mar. 4, 2016

Related U.S. Application Data
(60) Provisional application No. 61/874,502, filed on Sep. 6, 2013.

(51) Int. Cl. A01K 1/015; B01J 20/12; B01J 20/32; A61L 9/014

(52) U.S. Cl. CPC A01K 1/0154; A01K 1/0155; A61L 9/014; B01J 20/12; B01J 20/3204

(57) ABSTRACT

An anti-odor material for animal litters derived from the use of an odor-retardant agent including phosphorotriamides in powder form, and related process of manufacture. The anti-odor material may include a particulate support; and a crystallized layer of the odor-retardant agent coating a surface of the particulate support. The process for producing the anti-odor material may include heating the odor-retardant agent in a powder form to at least an odor-retardant agent melting point Tₘ to melt the powder form and produce a melted odor-retardant agent; mixing the melted odor-retardant agent with the particulate support for association thereof to produce the anti-odor material; and cooling the anti-odor material to a temperature below the odor-retardant agent melting point Tₘ to transform the melted odor-retardant agent into a crystallized odor-retardant agent. The anti-odor material may be mixed with an absorptive substrate to produce the animal litter.

### FIELD OF THE INVENTION

The present invention relates to an anti-odor material for animal litters. More particularly, the present invention relates to the use of phosphorotriamides in a powder form as an odor-retardant agent for animal litters.

### BACKGROUND OF THE INVENTION

The development of odors in soiled animal litters is a problematic phenomenon in relation to the degradation of specific compounds found in excretions. These compounds include ammonium, uric acid and urea, which degradation results in disruptive odors emanating from the litter immediately and over time. For example, urea is broken down over time into carbon dioxide and ammonia, the latter being a relatively volatile odorous compound.

Clumping animal litters have been developed so as to enable the formation of clumps upon contact with excretions, which are easily removable from a litter box. However, the clumps have to be removed very frequently to ensure not to emanate any undesirable odors.

A well-known solution in the art relates to the association of anti-odor agents with traditional litters or clumping litters. For example, chemical perfumes and essential oils have been used to mask the odors by exhibiting a pleasant smell. Additionally, disinfectants and antibacterial agents, such as boric acid and/or borax, enable to reduce or prevent the development of bacteria.

One approach is to use a urease inhibitor as odor-retardant agent. For example, the international patent application WO2011134074 discloses a dust and anti-odor animal litter including an odor-neutralising and dust-control agent associated with a substrate, and also an odor-retardant agent associated with the substrate. The odor-retardant agent may be a urease inhibitor, such as N-(n-butyl)thiophosphoric triamide in solution. The N-(n-butyl)thiophosphoric triamide (n-BTPT) is a urease inhibitor which inhibits the hydrolysis of urea into carbon dioxide and ammonia. WO2011134074 discloses applying n-BTPT as a solution including a solvent such as propyl glycol and between about 15% to 30% w/w of n-BTPT. The use of solutions for application of the urease inhibitor on the litter has some drawbacks and challenges related to the handling of the solvents and preparation of the multi-component solution. Solutions of n-BTPT may also have a viscosity which is sensitive to temperature change and the n-BTPT can undesirably crystallize in the solution. Another drawback of applying n-BTPT in a solution form is a clumping litter might clump readily upon contact with the said solution during the manufacturing process.

In summary, there is still a need for an improved technology for odor reduction in products such as animal litter.

### SUMMARY OF THE INVENTION

In one aspect of the present invention, there is provided a process for producing an anti-odor material including:
providing an absorptive substrate;
mixing an odor-retardant agent in a powder form with the absorptive substrate for producing the anti-odor material.

In an optional aspect, the process may include grinding the odor-retardant to the powder form prior to mixing with the absorptive substrate.

In another optional aspect, the process may include melting the odor-retardant agent in a powder form to produce a melted odor-retardant agent, so as to associate the melted odor-retardant agent with a surface of the absorptive substrate to produce the anti-odor material.

In another optional aspect of the process, the mixing may be performed during a mixing time which is sufficient to enable the coating of a surface of the absorptive substrate with a layer of the melted odor-retardant agent.

In another optional aspect, the process may include heating the absorptive substrate prior to mixing with the odor-retardant agent in the powder form, so as to melt the odor-retardant agent by contact with the heated absorptive substrate during mixing.

In another optional aspect, the process may include cooling the anti-odor material to transform the melted odor-retardant agent into a crystallized odor-retardant agent, after mixing with the absorptive substrate.

In another aspect of the present invention, there is provided a process for producing an anti-odor material for use in animal litter, the process including:
providing an absorptive substrate; and
mixing an odor-retardant agent in a melted phase with the absorptive substrate for producing the anti-odor material.

In an optional aspect, the process may include melting the odor-retardant agent in a powder form before mixing with the absorptive substrate, so as to obtain the odor-retardant agent in the melted phase. The melting may optionally be performed by heating the odor-retardant agent in powder form at a heating temperature between 50° C. and 80° C. Further optionally, the heating temperature may be between 68° C. and 75° C., further optionally, the heating temperature may be 70° C.

In another optional aspect of the process, the melting may be performed by heating the odor-retardant agent in the powder form to at least an odor-retardant agent melting point Tₘ to form the odor-retardant agent in the melted phase.

In another optional aspect of the process, the mixing may be performed during a mixing time which is sufficient to coat a surface of the absorptive substrate with a layer of the odor-retardant agent in the melted phase.

In another optional aspect, the process may include cooling the anti-odor material to a temperature below the odor-retardant agent melting point Tₘ to transform the melted phase of the odor-retardant agent into a crystallized phase.

In another aspect of the present invention, there is provided a process for producing an anti-odor material for use in animal litter, the process including:
providing a particulate support;
providing an odor-retardant agent in a powder form;
heating the odor-retardant agent in a powder form to at least an odor-retardant agent melting point Tₘ to melt the powder form and produce a melted odor-retardant agent;
mixing the melted odor-retardant agent with the particulate support for association thereof to produce the anti-odor material; and
cooling the anti-odor material to a temperature below the odor-retardant agent melting point Tₘ to transform the melted odor-retardant agent into a crystallized odor-retardant agent.

In an optional aspect of the process, the heating and mixing may be performed simultaneously.

In another optional aspect of the process, the heating of the odor-retardant agent in the powder form may be performed by a heating and mixing device, the device may including a rotary evaporator, a ribbon mixer or blender, a double ribbon mixer, a paddle mixer, a V blender, a double cone blender, a cone screw blender, an inclined mixer, a continuous mixer or blender and analog thereof.

In another optional aspect of the process, the mixing may be performed during a mixing time which is sufficient to coat a surface of the particulate support with a layer of the melted odor-retardant agent.

In another optional aspect of the process, the cooling of the anti-odor material may be performed continuously while mixing in cooling devices including a screw with a cooling section and analog thereof.

In another optional aspect, the process may include selecting the particulate support having a mesh size between 20 and 100. Optionally, the mesh size may be between 25 and 60.

In another optional aspect of the process, the odor-retardant agent may be N-(n-butyl)thiophosphoric triamide (n-BTPT), having the molecular formula C₄H₁₄N₃PS with the following structure:

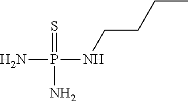

In another optional aspect, the animal litter releases an ammonia quantity in contact with urea, and the process may include providing n-BTPT in an adequate concentration in the animal litter so as to prevent between 80% and 100% of the ammonia quantity from being released.

In another aspect of the present invention, there is provided an anti-odor material for use in animal litter, the anti-odor material including an absorptive substrate and an odor-retardant agent in a powder form.

In an optional aspect of the anti-odor material, the absorptive substrate may include a non-clumping clay-based compound, a clumping clay-base compound, a limestone-based compound, a silica-based compound, a cellulose-based compound, a cellulose derivatives-based compound, an agricultural waste-based compound, a soil-based compound or a combination thereof.

In another aspect of the present invention, there is provided an anti-odor material for use in animal litter, the anti-odor material including:
a particulate support having a surface; and
an odor-retardant agent, the odor-retardant agent being associated with the surface of the particulate support.

In an optional aspect of the anti-odor material, the odor-retardant agent may be in a crystallized phase, the anti-odor material including a layer of crystallized odor-retardant agent coating the surface of the particulate support, the layer of crystallized odor-retardant agent resulting from the transition from a melted phase into a crystallized phase.

In another optional aspect of the anti-odor material, the odor-retardant agent may be physically absorbed at the surface of the particulate support to form an odor-retardant sub-surface region in the particulate support.

In another optional aspect of the anti-odor material, the odor-retardant agent may be adsorbed in pores of the surface of the particulate support to form an odor-retardant external layer on the surface of the particulate support.

In another optional aspect of the anti-odor material, the particulate support may include a plurality of particles which size and configuration is suited for use in animal litters. Optionally, said particles may be pellets and/or granules. Further optionally, the particles may have a mesh size between 20 and 100, optionally between 25 and 60.

In another aspect of the present invention, there is provided an anti-odor material for use in animal litter, the anti-odor material being prepared by a process including:
providing a particulate support;
providing an odor-retardant agent in a powder form;
heating the odor-retardant agent in the powder form to at least an odor-retardant agent melting point Tₘ to melt the powder form and produce a melted odor-retardant agent;
mixing the melted odor-retardant agent with the particulate support for association thereof to produce the anti-odor material; and
cooling the anti-odor material to a temperature below the odor-retardant agent melting point Tₘ to transform the melted odor-retardant agent into a crystallized odor-retardant agent.

In an optional aspect of the material, the particulate support may include an absorption compound, an adsorption compound or a combination thereof. Optionally, the absorption compound includes a clay-based compound, a cellulose-base compound, an agricultural waste-based compound, a soil-based compound or a combination thereof. Optionally, the adsorption compound includes a clay-based compound, a zeolite compound, a silica based compound, an activated carbon compound or a combination thereof. Further optionally, the clay-based compound includes bentonite, montmorillonite, arcillite, attapulgite or a combination thereof.

In an optional aspect of the material, the odor-retardant agent may include a urease inhibitor. Optionally, the urease inhibitor may includes a phosphorotriamide with the following molecular formula: R₁R₂R₃N₃PR₄wherein R₁, R₂ and R₃ are hydrogen atoms or alkyl groups, and R₄ is an oxygen or a sulfur atom. Further optionally, the phosphorotriamide may be N-(n-butyl)thiophosphoric triamide (n-BTPT), having the molecular formula C₄H₁₄N₃PS with the following structure:

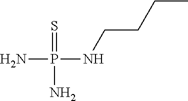

In another aspect of the present invention, there is provided a use of an odor-retardant agent in a powder form in an animal litter.

In another aspect of the present invention, there is provided a use of n-BTPT in a powder form as an odor-retardant agent in an animal litter.

In another aspect of the present invention, there is provided a use of n-BTPT in a crystallized phase obtained from a melted phase, as an odor-retardant agent in the manufacture of an anti-odor material for animal litters.

In another aspect of the present invention, there is provided an animal litter including:
an absorptive substrate; and
an anti-odor material including:
   a particulate support; and
   an odor-retardant agent, the odor-retardant agent being associated with the particulate support so as to coat a surface of the particulate support with a layer of odor-retardant agent;
wherein the odor-retardant agent is in a crystallized phase resulting from the transition from a melted phase into the crystallized phase.

In an optional aspect of the litter, the absorptive material may include a non-clumping clay-based compound, a clumping clay-base compound, a limestone-based compound, a silica-based compound, a cellulose-based compound, a cellulose derivatives-based compound, an agricultural waste-based compound, a soil-based compound or a combination thereof. Optionally, the particulate support may be made with the same material as the absorptive substrate.

In another optional aspect of the litter, the mass percentage of absorptive substrate with respect to the anti-odor material may be between 5 wt % and 40 wt % so as to obtain an equivalent pure n-BTPT content between 0.005 wt % and 0.05 wt %.

It should be understood that any one of the above mentioned optional aspects of each anti-odor material, related processes and related uses of an odor-retardant agent may be combined with any other of the aspects thereof, unless two aspects clearly cannot be combined due to their mutually exclusivity. For example, the various operational steps of the processes described herein-above, herein-below and/or in the appended Figures, may be combined with any of the material description appearing herein-above, herein-below and/or in the appended Figures.

### BRIEF DESCRIPTION OF THE DRAWINGS

Embodiments of the anti-odor material and related processes and uses according to the present invention are represented in and will be further understood in connection with the following figures.

FIG. 1 is a graph of the reduction in ammonia release expressed in percentage as a function of n-BTPT concentrations of grinded powder in the anti-odor material according to the present invention.

FIG. 2 is a graph of ammonia release expressed in part per million as a function of n-BTPT powder particle mesh size contained in the anti-odor material according to the present invention.

FIG. 3 is a graph of ammonia release expressed in part per million as a function of n-BTPT concentration contained in four samples of anti-odor material according to the present invention.

FIG. 4 is a ×55 scanning electron micrograph showing the surface of a particle of anti-odor material including a zeolite particulate support coated with 10% n-BTPT according to the present invention.

FIG. 5 is a ×600 scanning electron micrograph showing a portion of the surface of the anti-odor material of FIG. 4.

FIG. 6 is a ×300 scanning electron micrograph showing a portion of the cross-section of the anti-odor material of FIG. 4.

FIG. 7 is a ×100 scanning electron micrograph showing the surface of a particle of anti-odor material including a sodium bentonite particulate support coated with 20% n-BTPT according to the present invention.

FIG. 8 is a ×600 scanning electron micrograph showing a portion of the surface of the anti-odor material of FIG. 7.

FIG. 9 is a ×400 scanning electron micrograph showing a cross-section of the anti-odor material of FIG. 7.

FIG. 10 is a ×400 scanning electron micrograph showing a portion of the cross-section of the anti-odor material of FIG. 9.

FIG. 11 is a ×80 scanning electron micrograph showing the surface of a particle of anti-odor material including an arcillite particulate support coated with 40% n-BTPT according to the present invention.

FIG. 12 is a ×600 scanning electron micrograph showing a portion of the surface of the anti-odor material of FIG. 11.

FIG. 13 is a ×600 scanning electron micrograph showing a portion of the cross-section of the anti-odor material of FIG. 11.

FIG. 14 is a ×85 scanning electron micrograph showing the surface of a particle of anti-odor material including a montmorillonite particulate support coated with 40% n-BTPT according to the present invention.

FIG. 15 is a ×400 scanning electron micrograph showing a portion of the cross-section of the anti-odor material of FIG. 14.

While the invention will be described in conjunction with example embodiments, it will be understood that it is not intended to limit the scope of the invention to such embodiments. On the contrary, it is intended to cover all alternatives, modifications and equivalents as may be included as defined by the present description. The advantages and other features of the present invention will become more apparent and be better understood upon reading of the following non-restrictive description of the invention, given with reference to the accompanying figures.

### DETAILED DESCRIPTION

The present invention provides an anti-odor material and related process of manufacture including the use of an odor-retardant agent in a powder form. The anti-odor material is suited for use as animal litter which are soiled by odorous animal excretions. The presence of an odor-retardant agent in the material may decrease or minimize the development of further odorous compounds from excretions.

It should be understood that the odor-retardant agent refers to any agent retarding the formation of odorous volatile compounds by blocking an enzyme or reaction that would transform a less-odorous compound into volatile odorous compounds. For example, as urine includes urea, the urea has the potential to break down and form ammonia which is odorous. The odor-retardant agent, such as a urease inhibitor, can retard the formation of ammonia that results from the hydrolysis of urea, by blocking or retarding urease.

It should be understood that the powder form of the odor-retardant agent refers to a granular odor-retardant agent including fine particles having a mesh size from 12 to 300. The powder form of the odor-retardant agent may have the tendency to form clumps when stored.

According to one aspect of the present invention, the anti-odor material includes an absorptive substrate and an odor-retardant agent in a powder form. The absorptive substrate may be provided as particles suited for use as animal litter. The particles of absorptive substrate include pellets, granules, or any particles having size and configuration depending on the absorptive substrate use and its process of manufacture. The mass ratio of odor-retardant agent in powder form with respect to the absorptive substrate may be selected such that the anti-odor material is used as animal litter, for example cat litter. The powder of odor-retardant agent may be advantageously mixed with any absorptive substrates of animal litter to retard hydrolysis of urea by urease and form odorous ammonia. The use of odor-retardant agent in powder form presents several advantages in comparison to the use of odor-retardant agent in solution when manufacturing animal litter including an anti-odor material.

According to another aspect of the present invention, the anti-odor material may include a particulate support associated with the odor-retardant agent, such that the particulate support is coated with a layer of crystallized odor-retardant agent. The crystallized layer is derived from the crystallization of a layer of odor-retardant agent in melted phase. More particularly, after cooling of the anti-odor material, the melted phase of the odor-retardant agent transforms into a crystallized phase, thereby obtaining a particulate support coated with a layer of crystallized odor-retardant agent. The quantity of odor-retardant agent surrounding the particulate support may also be referred to as an odor-retardant agent charge.

It should be understood that the odor-retardant agent may be provided in powder form and that the melted phase of the odor-retardant agent is derived from the melting of the powder form of the odor-retardant agent.

It should be understood that the nature of the association between the particulate support and the odor-retardant agent may depend on the physical and chemical properties of the chosen particulate support with respect to the odor-retardant agent. For instance, the odor-retardant agent may be absorbed and/or adsorbed at the surface of the particulate support. It should be further understood that the quantity of odor-retardant agent that may be coated on the particulate support, also referred herein as the odor-retardant agent charge, may depend on the physical and chemical properties of the chosen particulate support, and also on the coating process during manufacture of the anti-odor material. Optionally, the odor-retardant agent may be associated with a surface of the particulate support so as to create an odor-retardant sub-surface region on the anti-odor material. Further optionally, the odor-retardant agent may be associated with a surface of the particulate support so as to create an exterior layer of odor-retardant agent on the anti-odor material.

According to another aspect of the present invention, there is provided an animal litter including an absorptive substrate and an anti-odor material. The anti-odor material includes a particulate support coated with an odor-retardant agent. The mass ratio of anti-odor material with respect to the absorptive substrate may be selected so as to reach a given concentration of odor-retardant agent in the animal litter, or such that the resulting animal litter meets a specific ammonia release criteria. The selected mass ratio of anti-odor material with respect to the absorptive substrate may therefore depend on the odor-retardant agent charge of the particulate support. The particles of anti-odor material may be advantageously mixed with any absorptive substrates of animal litter to retard hydrolysis of urea by urease and form odorous ammonia. The use of particulate support coated with the odor-retardant agent enables to increase or maximize the availability of the odor-retardant agent for contacting the excretions in the litter. The use of particulate support coated with the odor-retardant agent may also facilitate the homogeneity of the anti-odor material distribution in the animal litter.

In an optional aspect of the present invention, the absorptive substrate as described herein may be used as the particulate support.

In an optional aspect of the present invention, the absorptive substrate may be selected according to its absorption capacity in relation to animal excretions. The absorptive substrate may include any existing substrates for animal litter, such as non-clumping clay-based compound, clumping clay-based compound, limestone-based compound, silica-based compound, cellulose-based compound, cellulose derivatives-based compound, agricultural waste-based compound, soil-based compound or a combination thereof.

In another optional aspect of the present invention, the particulate support may be selected according to its association capacity in relation to the odor-retardant agent. Depending on the physical and chemical properties of the selected particulate support, the odor-retardant agent in a melted phase may be physically absorbed, adsorbed or a combination thereof. The particulate support may include a plurality of pores that receive the melted odor-retardant agent. The particulate support may include clay-based compound, zeolite-based compound, activated carbon-based compound, silica-based compound, cellulose-based compound, cellulose derivatives-based compound, agricultural waste-based compound, soil-based compound or a combination thereof.

Optionally, the clay-based compound may include montmorillonite, bentonite, attapulgite, arcillite or a combination thereof. Optionally, the agricultural waste-based compound may include corn cobs and/or wheat which have been crushed or granulated. The Examples provided hereinafter includes experiments on specific particulate support and absorptive substrate. It should be understood that the present invention is not limited to those specific examples.

It should be understood that bentonite refers to sodium bentonite, montmorillonite refers to calcium bentonite and arcillite refers to calcined calcium bentonite.

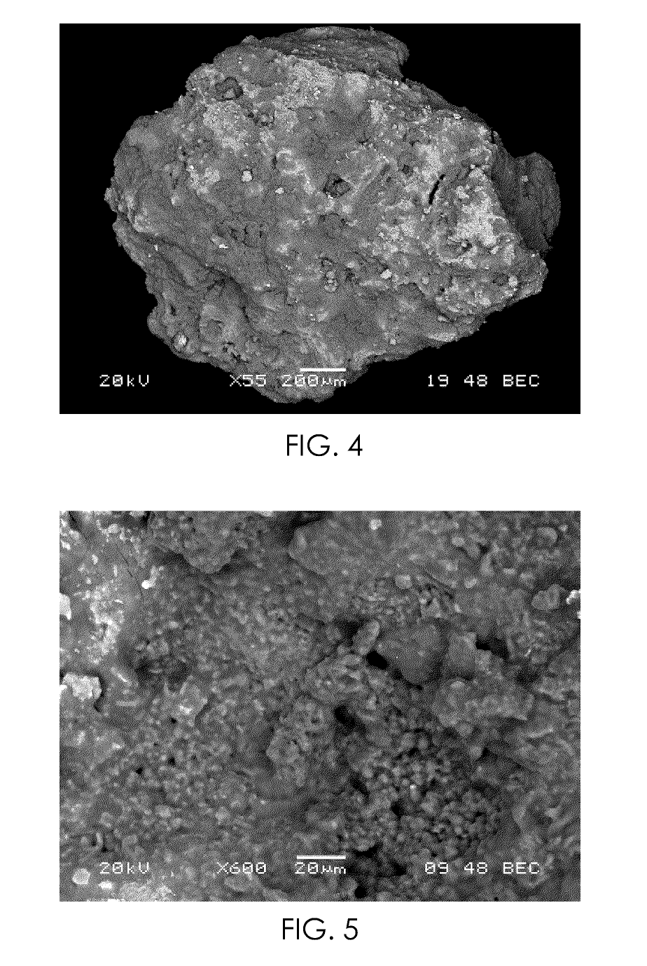

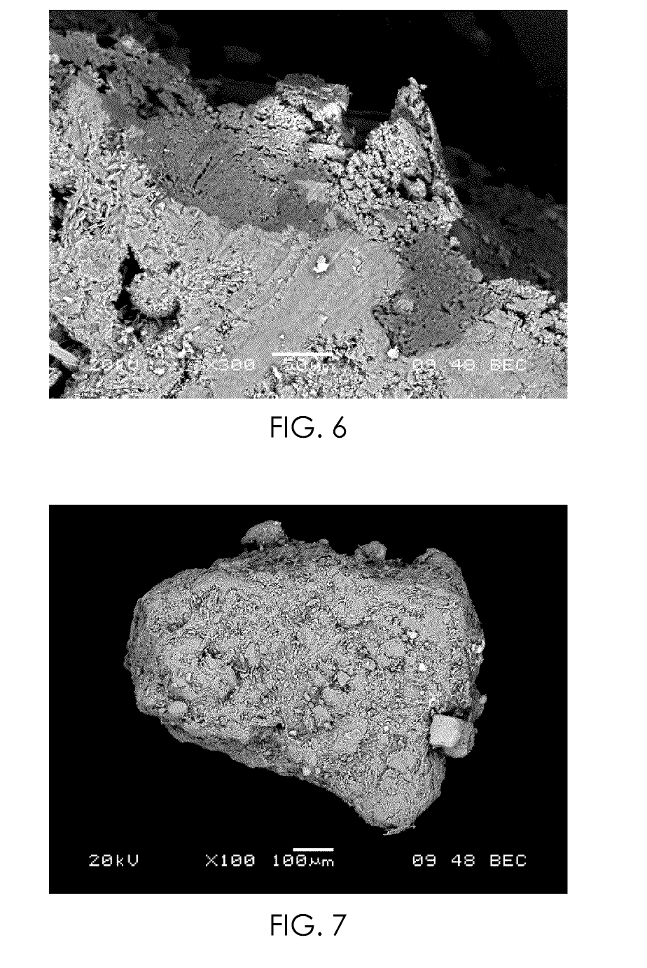

Scanning electron micrographs showing the surface of particulate support coated with crystallized n-BTPT are shown in FIGS. 4-5, 7-8, 11-12 and 14. Scanning electron micrographs showing cross sections of these same coated particulate supports are shown in FIGS. 6, 9, 10, 13 and 15. The scanning electron microscope used was a low vacuum JEOL JSM 5900.

FIGS. 4 and 5 show the surface of a porous zeolite particle including 10% of n-BTPT. As can be seen, the surface of the zeolite particulate support is not evenly coated with crystallized n-BTPT. Referring to FIG. 6, the cross section of the support includes pores irregularly filled with crystallized n-BTPT.

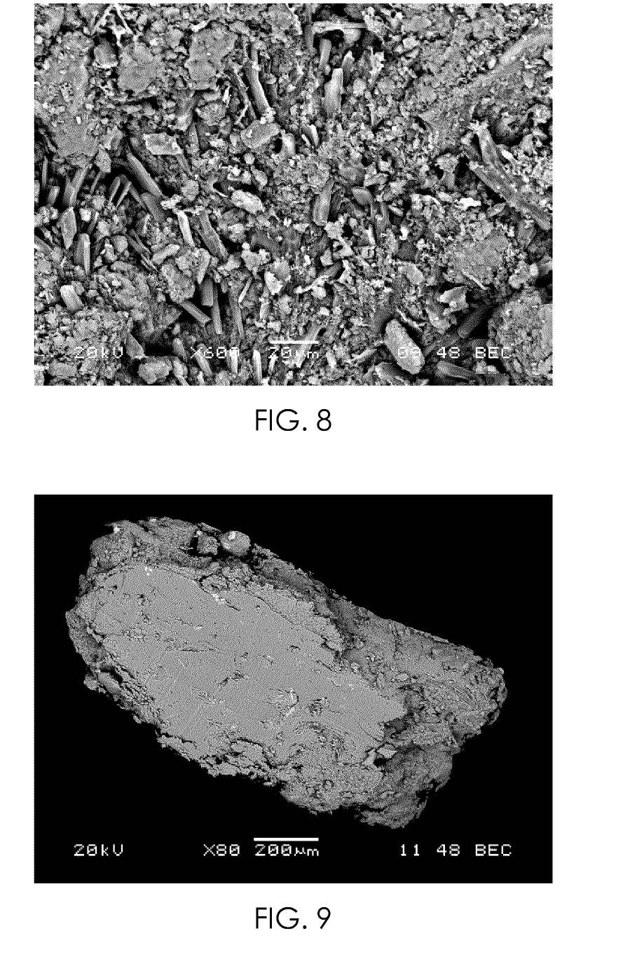

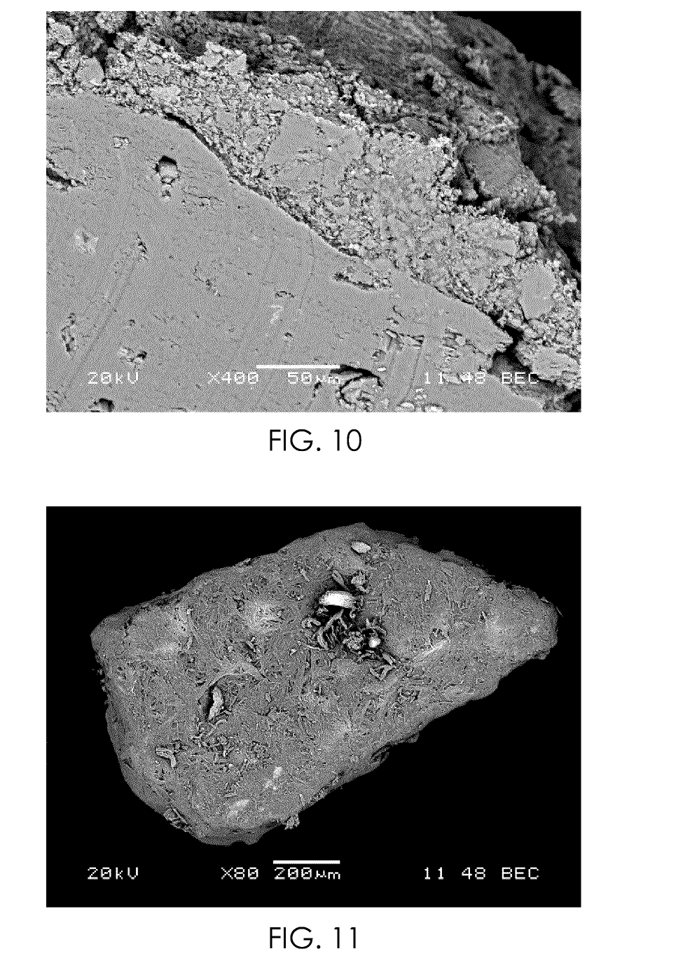

FIGS. 7 and 8 show the surface of a sodium bentonite particle including 20% of n-BTPT. As can be seen, crystals of n-BTPT were formed at the surface of the particulate support nut the content of 20% of n-BTPT does not enable a full coverage of the particulate support. Referring to FIGS. 9 and 10, the cross section of the support shows an uneven coating of the support including portions without n-BTPT.

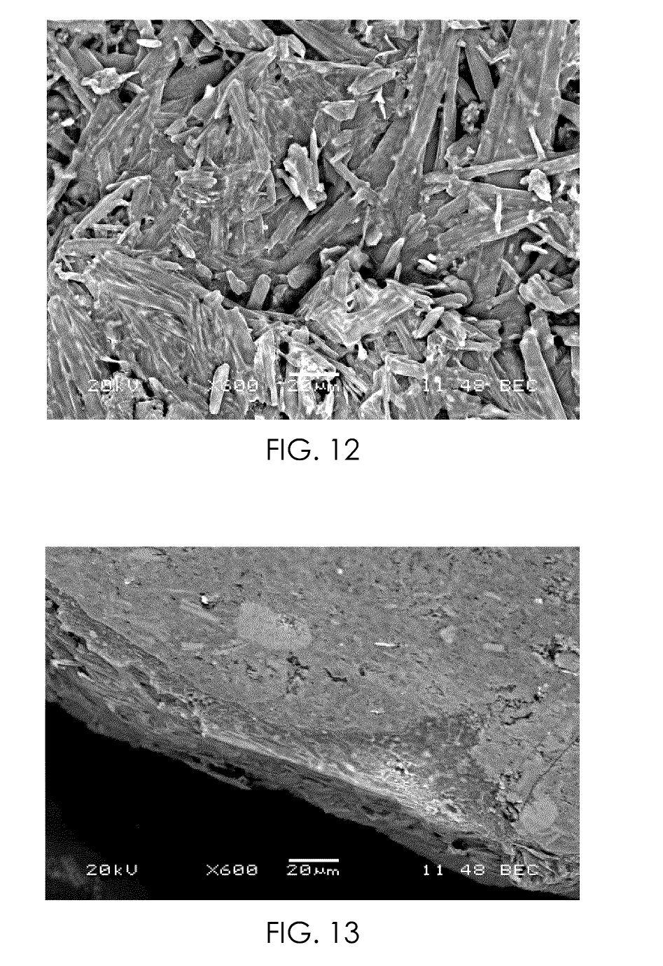

FIGS. 11 and 12 show the surface of an arcillite particle including 40% of n-BTPT. The surface of the support is fully coated with a layer of crystallized n-BTPT. The cross section of FIG. 13 also shows that a thin layer of n-BTPT covers inorganic compounds of the bentonite particle.

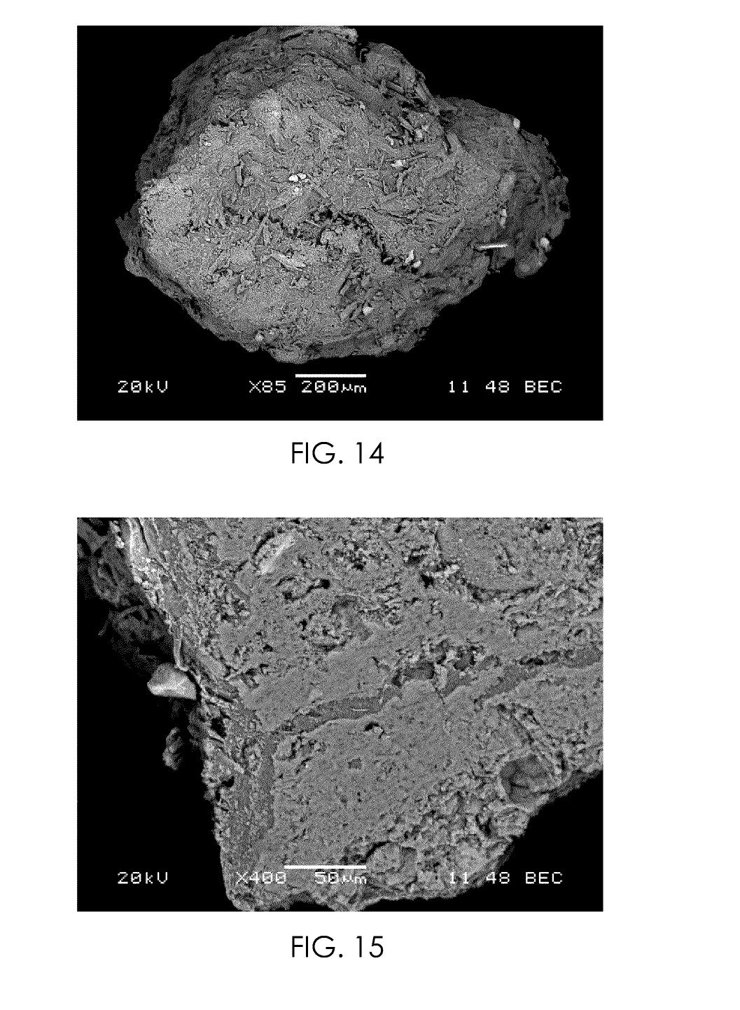

FIG. 14 shows the surface of a montmorillonite particle including 40% of n-BTPT. The surface of the support is fully coated with a layer of crystallized n-BTPT. The cross section of FIG. 15 also shows that a thin layer of n-BTPT covers inorganic compounds of the montmorillonite particle and even that n-BTPT may be found in internal pores or structures of the particle (also referred to as sub-surface region).

It should be understood that the term “coated” or “coating” refers to any association of the odor-retardant agent with an active surface of the particulate support. Depending on the nature of the particulate support and the amount of odor-retardant agent in the anti-odor material, the particulate support may be fully coated with a layer of crystallized odor-retardant agent or may only include scattered pockets of crystals of odor-retardant agent. The odor-retardant agent may be therefore found in the sub-surface region of the anti-odor material and/or as an exterior layer of the material.

In an optional aspect of the present invention, the odor-retardant agent may be a urease inhibitor. Optionally, the urease inhibitor may be one or more phosphorotriamides with the following molecular formula: R₁R₂R₃N₃PR₄ wherein R₁, R₂ and R₃ are hydrogen atoms or alkyl groups, and R₄ is an oxygen or a sulfur atom. In some aspects, the phosphorotriamides may include cyclohexyl thiophosphoric triamide (CHTPT), cyclohexyl phosphoric triamide (CHPT), N-(n-butyl)phosphoric triamide (n-BTPT), N-aliphatic phosphoric triamide, N,N-aliphatic phosphoric triamide and combination thereof.

Optionally, the urease inhibitor may be n-BTPT in powder form with the following formula:

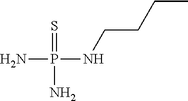

As will be demonstrated in further below examples, it has been found that the use of an odor-retardant agent in powder form or coated on particulate support can further limit ammonia release from excretions in comparison to the use of an odor-retardant agent in solution mixed with the absorptive substrate.

For example, solutions of n-BTPT can reduce the availability of n-BTPT molecules to urea contained in excretions in comparison to powder n-BTPT. Powder of n-BTPT as an odor-retardant agent may be advantageously mixed with any absorptive substrates of animal litter to retard hydrolysis of urea by urease and form odorous ammonia. It should be understood that the powder of n-BTPT as referred herein has a n-BTPT content of 95% to 99%. Optionally, n-BTPT in powder form may be added to the absorptive substrate to produce the animal litter with a mass ratio ranging from 0.080 Kg of n-BTPT per ton of litter to 0.450 Kg of n-BTPT per ton of litter. Further optionally, n-BTPT may be added to the animal litter with a mass ratio of 0.230 Kg of n-BTPT per ton of litter. It should be noted that the anti-odor material according to the present invention may reduce the ammonia release from 50 to 100% after 48 hours of contact with excretions.

In an optional aspect of the present invention, the odor-retardant agent in the powder form is made of particle having a mesh size between 12 and 300, further optionally between 40 and 300. Those fine particles of odor-retardant agent may be further mixed with particles of the absorptive substrate according to an aspect of the present invention. Additionally, the quantity of n-BTPT in powder form to be added to the absorptive substrate may be selected so as to reach a n-BTPT concentration in the animal litter between 0.005 and 0.05 wt % (in pure n-BTPT equivalent with respect to the total weight of the animal litter).

In another optional aspect of the present invention, the particulate support may include small particles having an average mesh size of at most 325, and further optionally between 40 and 100. More particularly, the particulate support may have a size distribution including 0 to 2 wt % of the particles having an average mesh size inferior to 8, 1 to 3 wt % of the particles having an average mesh size inferior to 12, 20 to 25 wt % of the particles having an average mesh size inferior to 16, 55 to 65 wt % of the particles having an average mesh size inferior to 30, 8 to 20 wt % of the particles having an average mesh size inferior to 60 and the rest of the particles having an average mesh size between 100 and 300. Optionally, clay-based particulate support including bentonite or montmorillonite may have a mesh size distribution as provided in the below Table.

| Sieve (mesh) | Bentonite % | Montmorillonite % |
|---|---|---|
| 10 | 0.05 | 0 |
| 12 | 0.05 | 0 |
| 25 | 1.00 | 43.48 |
| 40 | 37.69 | 43.74 |
| 60 | 39.89 | 12.18 |
| 100 | 12.60 | 0.20 |
| Pan | 8.72 | 0.40 |

It should be understood that the present invention is not limited to a precise size distribution of the particulate support and the anti-odor material may include various particulate support. According to the mesh size of the particulate support, the anti-odor material may be used as additive for small domestic animal litter, such as litters for cats and dogs. Bigger particles may be included in livestock litter particles, such as litters for pigs, cows and horses.

In another optional aspect of the present invention, the animal litter may include particles of absorptive substrate mixed with support particles coated with a layer of crystallized n-BTPT. The quantity of anti-odor material to be included in the animal litter is to be selected according to the odor-retardant agent charge of the particulate support and the desired odor-retardant agent concentration in the litter (or desired ammonia release in ppm). Optionally, according to the nature of the particulate support and the process of manufacture of the anti-odor material, the odor-retardant agent charge on the particulate support may be between 5 and 80 wt % of the total weight of anti-odor material, further optionally between 20 and 50 wt % of the total weight of anti-odor material. For instance, n-BTPT may be added to the support with a mass concentration between 5 wt % and 50 wt %, optionally between 25 wt % and 45 wt %, and further optionally of 40 wt %, with respect to the total mass of the anti-odor material. Depending on the n-BTPT charge of the anti-odor material, the mass percentage of the anti-odor material to be added to the absorptive substrate to form the animal litter may be selected so as to reach a n-BTPT concentration in the litter between 0.005 and 0.05 wt % (in pure n-BTPT equivalent with respect to the total weight of the litter).

The present invention further relates to a process for producing the anti-odor material. The process steps may vary according to the embodiments of the produced anti-odor material. More particularly, the process steps to produce the anti-odor material including the odor-retardant agent in powder form may differ from the process steps to produce the anti-odor material including a particulate support coated with the odor-retardant agent. The use of the powder form of the odor-retardant agent is advantageous in various embodiments of the process. In one aspect of the present invention, there is provided a process for producing an anti-odor material including providing an absorptive substrate; and mixing an odor-retardant agent in a powder form with the absorptive substrate, thereby obtaining the anti-odor material. Optionally, the process may include grinding the odor-retardant agent to the powder form before mixing with the absorptive substrate. Embodiments of this process are suited for example to produce the anti-odor material including the absorptive substrate and the odor-retardant agent in powder form.

In another aspect of the present invention, there is provided a process for producing an anti-odor material including providing a particulate support; and mixing an odor-retardant agent in a melted phase with the particulate support. Embodiments of this process are related to the use of the odor-retardant agent, such as n-BTPT, in a melted phase so as to coat the particulate support during mixing with the same. In an optional aspect of the process, an absorptive substrate as described above is used as particulate support and the process may include melting the odor-retardant agent in the powder so as to further associate the melted odor-retardant agent with a surface of the absorptive substrate to produce the anti-odor material.

In another aspect of the present invention, there is provided a process for producing an anti-odor material including providing a particulate support and an odor-retardant agent in a powder form. The process further includes heating the odor-retardant agent in the powder form to at least an odor-retardant agent melting point Tₘ to melt the powder form and produce a melted odor-retardant agent. The process also includes mixing the melted odor-retardant agent with the particulate support for association thereof to produce the anti-odor material. The final step of the process includes cooling the anti-odor material to a temperature below the odor-retardant agent melting point Tₘ to transform the melted odor-retardant agent into a crystallized odor-retardant agent.

Optionally, the process may include melting the odor-retardant agent in the powder form before mixing with the particulate support. Further optionally, the process may include melting the odor-retardant agent in the powder form during mixing with the particulate support. In both case, the particulate support is coated with a layer of melted odor-retardant agent. After cooling, the layer of melted odor-retardant agent transforms into a layer of crystallized odor-retardant agent such that the produced anti-odor material is ready for use in animal litters.

It should be understood that the melted phase of the odor-retardant agent refers to the state of the odor-retardant which is obtained by melting a powder form (solid) of the odor-retardant agent. Therefore, the melted phase is only made of the odor-retardant agent and differs from a solution of the odor-retardant, which includes a solvent. The melting of the odor-retardant agent may optionally be performed by heating the odor-retardant agent in the powder form to at least the odor-retardant agent melting point Tₘ.

Optionally, in case of using the n-BTPT as the odor-retardant agent, the melting may be performed by heating n-BTPT in powder form at a temperature between 50° C. and 80° C., optionally between 68° C. and 75° C. and further option ally at 70° C.

It should be further understood that the crystallized phase refers to the state of the odor-retardant agent which is obtained after transition from the melted phase by crystallization. The crystallization of the odor-retardant material may optionally be performed by cooling to a temperature below the odor-retardant agent melting point Tₘ.

In an optional aspect of the process, the particulate support (or absorptive substrate used as support) may be heated prior to mixing with the odor-retardant agent in the powder form. The melting of the odor-retardant agent may be thereby obtained by contact with the heated absorptive substrate during mixing.

In another optional aspect of the process, the mixing step may be performed during a mixing time which is in accordance with the desired odor-retardant agent charge of the anti-odor material. For a given weight of particulate support, the mixing time may therefore be selected so as to coat a surface of the particulate support with a layer of crystallized odor-retardant agent having a desired thickness.

It should be understood that each step of the process may be adapted and tailored so as to produce an anti-odor material according to the above-described embodiments.

In an optional aspect of the process, the mixing step may be performed in a mixing device, including various mixers or blenders known in the art of mixing, so as to obtain a homogeneous mixing.

Optionally, the powder of odor-retardant agent may be added continuously by means of a screw while particles of the absorptive substrate are falling through or while the absorptive substrate is being transported on a conveyor or by any other means resulting in the contact of the powder of odor-retardant agent with the absorptive substrate. An homogeneous distribution of the powder of odor-retardant agent, such as n-BTPT, within the particles of absorptive substrate may be obtained. Alternatively, the mixing device may include a Rollo-mixer®, a vertical mixer or a dosing apparatus.

Further optionally, the particulate support may be coated with the odor-retardant agent by using a heating mixer or blender. The odor-retardant agent may be melted and simultaneously mixed with the particulate support so as to coat the latter with a layer of the melted odor-retardant agent. The process optionally includes adding the odor-retardant agent in a powder form to the particulate support in the mixing device, and then rising a mixing temperature to the melting temperature of the odor-retardant agent, so as to melt the odor-retardant agent for association with the particulate support. Related heating and mixing devices include a rotary evaporator, a ribbon mixer, a double ribbon mixer, a paddle mixer, a V blender, a double cone blender, a cone screw blender, an inclined mixer, a continuous blender/mixer and any analog thereof.

Further optionally, as the particulate support may be mixed with the odor-retardant agent in a melted phase, the odor-retardant agent may be melted prior to the mixing step. The mixing may be performed by injecting the odor-retardant agent in a melted phase in a mixing device, fluidized bed reactor or analogs thereof for ensuring contacting and mixing with the particulate support. For example, the melted n-BTPT may be added via vertical or horizontal injection nozzles to a horizontal or vertical bed of particulate support.

Further optionally, the cooling of the anti-odor material may be performed continuously while mixing in adapted devices, such as a screw with a cooling section and analog thereof, or in a discontinuous way, by putting the above-mentioned mixing devices in cooling conditions (using a cold fluid) or by transferring the anti-odor material in a dryer. Optionally, the anti-odor material may also merely be cooled at ambient temperature.

In another optional aspect of the present invention, the process may include mixing the anti-odor material, including the odor-retardant agent coated particulate support, with particles of absorptive substrate so as to obtain a litter suited for use as cat litter for instance.

Advantageously, the use of the odor-retardant agent, such as n-BTPT, in powder form can enhance reliability because of the stability and density of the powder under various process conditions. Furthermore, during transportation of the odor-retardant agent to the site of production, powder is less sensitive to temperature change than some solutions including odor-retardant agent. Problems may be encountered when using solutions of odor-retardant agent, as the solution viscosity may vary according to the temperature and crystallisation may occur under certain conditions. Additionally, the use of powder enables avoiding the use of solvents, some of which may have various drawbacks. Furthermore, the melting of the powder form of the odor-retardant agent enables to obtain a melted phase which is used to coat a particulate support. After cooling, the particulate support is coated with a crystallized layer of odor-retardant agent, such as n-BTPT, increasing the contact surface with the excretions and increasing the reduction of ammonia volatilization.

Some embodiments of the present invention are illustrated by the following examples.

### EXAMPLES

Experiments with the odor-retardant agent in powder form or in melted phase have been performed to show the efficiency of the anti-odor material which was prepared accordingly.

Experiments have been performed so as to evaluate the dosimetric responses regarding the detection of ammonia released by anti-odor materials.

### Example 1

### n-BTPT Powder

To simulate soiled anti-odor material, urea/urease solution was prepared and added to the anti-odor material.

Material:

100 ml beaker;

Plastic bowl with cover (hole in the cover);

Precision balance 0.1 g, with a capacity of 2000 g;

Timer;

Urea in powder, 98+%, Sigma®;

Urease in powder, 20990 unit/g solid, Sigma®;

Gastec pump GV-100S;

Detection tube type 3L (0-50 ppm);

Precision balance 0.001 g, capacity 150 g; and

Standard volume for dosimetry.

Preparation of Urea/Urease Solution:

Rinse the beaker with demineralised water.

Weight 7.5 g of urea.

In the same beaker, weight 50 g of demineralised water.

In a small plastic cup, weight 0.012 g of urease. Then empty the plastic cup in the beaker containing the water and rinse it with the water/urea solution.

Agitate carefully until total dissolution.

Preparation of Soiled Anti-Odor Material Samples:

Fill a standard volume with anti-odor material (litter+n-BTPT powder) and equalize the surface. Empty the standard volume in a plastic bowl used for analysis.

Empty slowly the urea/urease solution on the anti-odor material while making sure that no solution comes in contact with sides of the plastic bowl.

Put a cover on the plastic bowl (the cover having a hole).

Dosimetry Analysis of the Soiled Anti-Odor Material Samples:

Insert the tube of a Gastec pump (GV-100S) in the hole of the cover.

Note the start time of the test and take a reading of the tube every 30 minutes for the first 7 hours.

Take a reading at 24 and 48 hour.

Dosimetry tests were performed according to the methodology above. The results were taken after 48 h of testing.

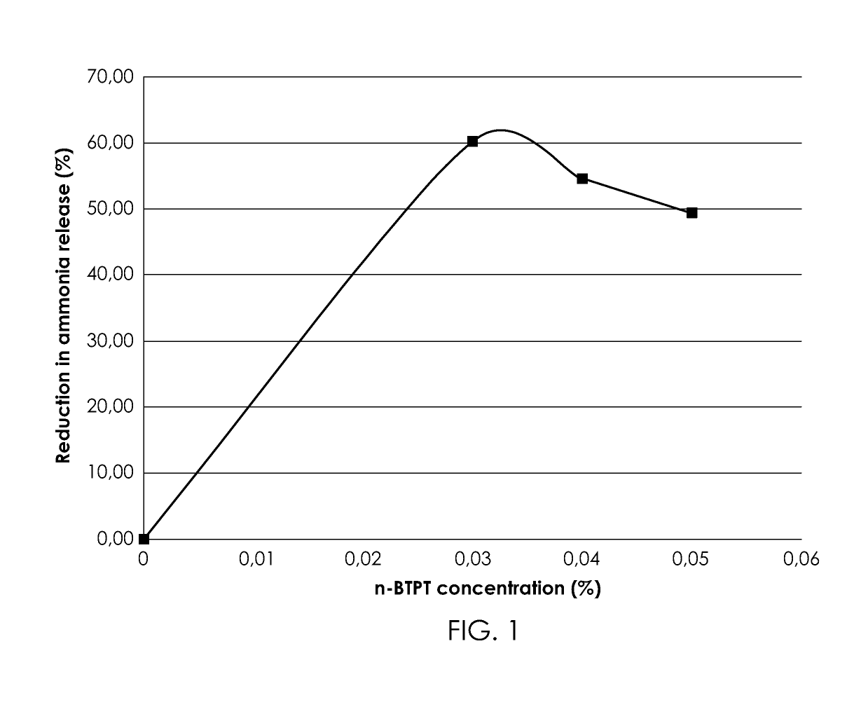

Referring to FIG. 1, at a n-BTPT concentration of 0.03%, the ammonia release reaches a maximum of 62%. For a n-BTPT concentration exceeding 0.03%, there is no beneficial effect regarding the ammonia release when increasing the n-BTPT concentration.

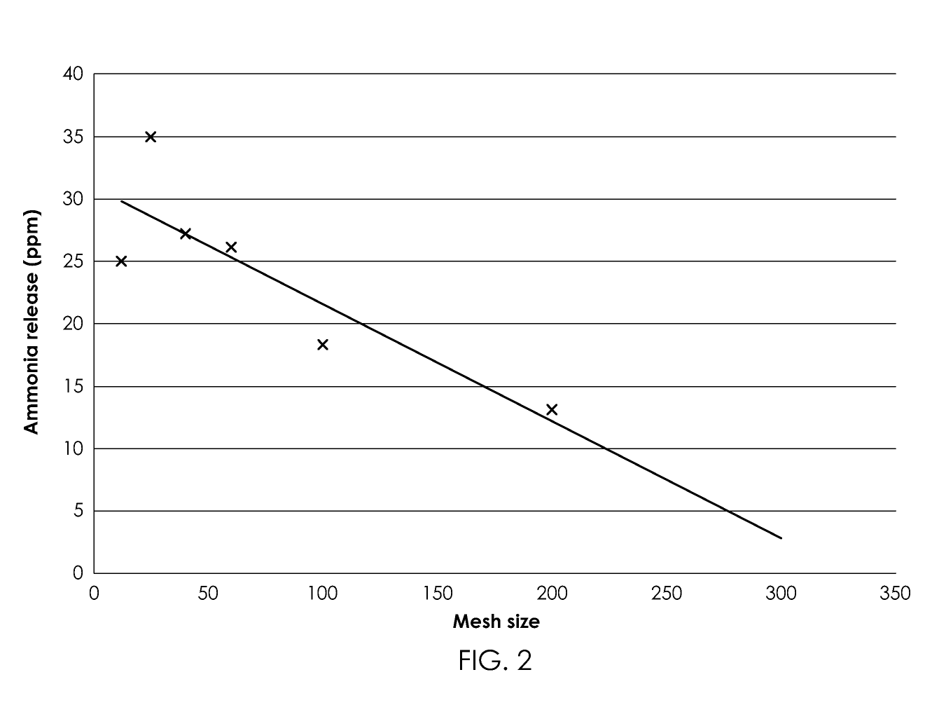

FIG. 2 illustrates the ammonia release in parts per million as a function of n-BTPT powder size distribution (in mesh size). As shown in FIG. 2, smaller particle size of n-BTPT increases the reduction of ammonia volatilization in parts per million. This result may be explained by a better dispersion of the powder of n-BTPT in association with the particles of absorptive substrate and therefore, an increased likelihood that a urea molecule will be in contact with said n-BTPT for inhibition of urea breakdown to ammonia.

### Example 2

### n-BTPT on Bentonite Particulate Support

Experiments have also been performed with particulate support associated with the odor-retardant agent n-BTPT and mixed with particles of bentonite (serving as absorptive substrate). The particulate support included bentonite or zeolite, as referred to in Table 1 below. The results provide the ammonia volatilization in parts per million in function of the concentration of melted n-BTPT as a percentage with respect to the total quantity of litter particles (support particles+particles of absorptive substrate).

TABLE 1

| | | 10% melted n- | 15% melted n- | 20% melted n- |
|---|---|---|---|---|
| | Powder | BTPT on | BTPT on | BTPT on |
| | nBTPT | bentonite (final | bentonite (final | bentonite (final |
| | 350 | concentration | concentration | concentration |
| | g/MT | 350 g/MT) | 350 g/MT) | 350 g/MT) |
| Ammonia | 25 | 25 | 24 | 19 |
| release | | | | |
| (48 h) | | | | |
| (ppm) | | | | |

### Example 3

### n-BTPT on Particulate Support

Further experiments have also been performed with particulate support associated with the odor-retardant agent and mixed with particles of bentonite (acting as absorptive substrate). The particulate support included bentonite, limestone, white zeolite or a combination thereof.

Preparation of Anti-Odor Material Samples:

The anti-odor material samples were prepared according to the following methodology:
the particulate support and n-BTPT were respectively weighted precisely;
the weighted particulate support was heated in a hot-water bath at 65-70° C.;
when the particulate support reached a temperature around 65° C., a small portion of the n-BTPT was poured on the hot particulate support for melting thereof;
the particulate support and n-BTPT were mixed so as to obtain an homogeneous distribution of the n-BTPT on the support;
the two previous step were reproduced until the total weighted quantity of n-BTPT is poured on the support;
the coated support was let to cool down so as to enable crystallization of the associated n-BTPT so as to form the anti-odor material; and
in case of any aggregates of anti-odor material, a spatula was used to break the aggregates.

The particulate support that was used for the preparation of the anti-odor material samples included limestone, zeolite and bentonite (see Table 2 below).

TABLE 2

| | | | | | | | bentonite |
|---|---|---|---|---|---|---|---|
| | | | | | | | (fines and |
| | | particulate | n- | | | | dust) and |
| | n-BTPT | support | BTPT | limestone | zeolite | bentonite | 10% |
| Sample # | (%) | (%) | (g) | (g) | (g) | 12 mesh | CaCO₃ |
| LAB-1 | 30 | 70 | 3 | | 7 | | |
| LAB-2 | 20 | 80 | 2 | | 8 | | |
| LAB-3 | 10 | 90 | 1 | | 9 | | |
| LAB-4 | 18 | 82 | 2 | 9 | | | |
| LAB-5 | 5 | 95 | 0.5 | | 9.5 | | |
| LAB-7 | 10 | 90 | 1 | | 9 | | |
| LAB-8 | 5 | 95 | 0.5 | | 9.5 | | |
| LAB-9 | 15 | 85 | 1.5 | | 8.5 | | |
| LAB-10 | 5 | 95 | 0.5 | 9.5 | | | |
| LAB-11 | 2.5 | 97.5 | 0.5 | 19.5 | | | |
| LAB-12 | 5 | 95 | 0.5 | | | 9.5 | |
| LAB-13 | 10 | 90 | 1 | | | 9 | |
| LAB-14 | 10 | 90 | 0.5 | | | | 9.5 |
| LAB-15 | 20 | 80 | 2 | | | | 8 |
| LAB-16 | 15 | 85 | 1 | | | | 9 |
| LAB-17 | 20 | 80 | 2 | | | | 8 |

*LAB-1 to LAB-5 were prepared with limestone and potassium aluminosilicate clinosilicate zeolite 8X (16 mesh)
**LAB-7 to LAB-11 were prepared with limestone sieved to at most 40 mesh and clinoptilolite zeolite sieved to at least 100 mesh.
***LAB-12 and LAB-13 were prepared with bentonite sieved between 16 and 40 mesh
****LAB-14 to LAB-17 were prepared with fines and dust from bentonite sieved to at least 25 mesh.

Observations on the Preparation of Anti-Odor Material Samples:

Samples LAB-1 and LAB-2 seem to be saturated in n-BTPT. The formed anti-odor material tend to aggregate while cooling and to stick to the mixer walls, leaving a white residue thereon (crystallized n-BTPT).

Sample LAB-3 is homogeneous and there is only a very small quantity of white residue on the mixer walls.

Sample LAB-4 (limestone) had too high humidity content and forms, after cooling, a large and hard agglomerate with a white residue thereon and on the mixer walls.

Sample LAB-5 including only 5% of n-BTPT is not homogeneously combined with the n-BTPT. Some support particles were not evenly coated with crystallized n-BTPT.

Sample LAB-7 is almost saturated with n-BTPT, there is no white residue on the mixer walls.

Sample LAB-9 shows that 15% of n-BTPT is too high for the zeolite support because a white residue starts to form on the mixer walls.

Therefore, a percentage of +/−10% of n-BTPT seems to be a suited quantity of odor-retardant agent to enable an even coating of zeolite particles.

Sample LAB-10 and LAB-11 show that limestone particles are not a support which is adequate for being coated with n-BTPT. A lot of n-BTPT is found on the mixer walls and the limestone particles are not evenly coated. Only a very thin layer could be deposited on the limestone support, which is too thin to obtain a proper reduction of ammonia volatilization.

Sample LAB-12 is not evenly coated with a layer of n-BTPT. A content of 5% of n-BTPT is insufficient to coat a bentonite support.

Sample LAB-13 is evenly coated with a melted layer of n-BTPT and the particles become darker before crystallization of the melted n-BTPT. There is no white residue on the mixer walls.

Sample LAB-16 is evenly coated with a melted layer of n-BTPT. Thus, a 15% content of n-BTPT seems to be suited for a bentonite support.

Preparation of the Absorptive Substrate

The absorptive substrate was prepared by mixing 90% of bentonite with 10% of limestone.

Preparation of Litter Samples:

The cooled anti-odor material was weighted according to a precise quantity to be added to 1 kg of absorptive substrate so as to obtain a 0.035% of n-BTPT in the final litter samples (see Table 3 below).

Dosimetry Tests:

Dosimetry tests were then realised to evaluate the performance of the litter samples by measuring the ammonia release (in ppm). More particularly, performances of the odor-retardant material with n-BTPT in a powder form, in melted phase or coating a particulate support could be evaluated.

Tables 3 to 7 provides dosimetry results for different litter mixtures (LIT) prepared according to the above methodology with the anti-odor material samples (LAB) provided in Table 2 or with pure n-BTPT in powder form or in a melted phase. As the series of experiments has not been performed on the same day, the dosimetry result for the litter sample of reference (REF) may differ from one Table to another (due to differences in temperature conditions, sample preparation, etc.).

TABLE 3

| | Mass of sample to be | | Urea/ |
|---|---|---|---|
| | added in 1 kg of absorptive | | Urease |
| Litter | substrate to obtain a | | dosimetry |
| mixture | litter 0.035% of n-BTPT | n-BTPT state | (ppm) |
| LIT-1 | 0.35 g of pure n-BTPT | powder | 28.30 |
| (REF) | | | |
| LIT-2 | 0.35 g of pure n-BTPT | melted | 27.20 |
| LIT-3 | 3.50 g of LAB-3 | coating a particle of | 34.00 |
| LIT-4 | 7.00 g of LAB-5 | zeolite (14 mesh) | 39.00 |

Observations:

The litter mixture LIT-1 is as performant as the litter mixture LIT-2. Therefore, the melted n-BTPT is equivalent to n-BTPT in powder form in terms of urea/urease dosimetry.

The melted n-BTPT supported on a coarse zeolite particle is however less performant than n-BTPT powder as odor-retardant material.

TABLE 4

| | Mass of sample to be | | Urea/ |
|---|---|---|---|
| | added in 1 kg of absorptive | | Urease |
| Litter | substrate to obtain a | | dosimetry |
| mixture | litter 0.035% of n-BTPT | n-BTPT state | (ppm) |
| LIT-5 | 3.50 g of LAB-7 | coating a particle | 27.20 |
| LIT-6 | 7.00 g of LAB-8 | of zeolite | 27.20 |
| LIT-7 | 2.33 g of LAB-9 | (>100 mesh) | 24.00 |
| LIT-8 | 7.00 g of LAB-10 | coating a particle | 30.00 |
| LIT-9 | 14.00 g of LAB-11 | of limestone | 24.00 |
| | | (25-40 mesh) | |
| LIT-10 | 0.35 g of pure n-BTPT | powder | 25.00 |
| (REF) | | | |

Observations:

The litter mixture LIT-7 provides the best reduction of ammonia volatilization, similar to the reference litter mixture LIT-10.

TABLE 5

| | Mass of sample to be | | Urea/ |
|---|---|---|---|
| | added in 1 kg of absorptive | | Urease |
| Litter | substrate to obtain a | | dosimetry |
| mixture | litter 0.035% of n-BTPT | n-BTPT state | (ppm) |
| LIT-11 | 7.00 g of LAB-12 | coating a particle | 23.23 |
| LIT-12 | 7.00 g of LAB-13 | of bentonite | 33.29 |
| LIT-13 | 0.35 g of pure n-BTPT | powder | 15.48 |
| (REF) | | | |

Observations:

The litter mixtures LIT-11 and LIT-12 are not as efficient as pure n-BTPT powder in terms of reduction in ammonia volatilization. A higher content in n-BTPT does not guarantee a better reduction in ammonia release as the performance of the litter mixture LIT-12 is inferior to the one of the litter mixture LIT-11.

TABLE 6

| | Mass of sample to be | | Urea/ |
|---|---|---|---|
| | added in 1 kg of absorptive | | Urease |
| Litter | substrate to obtain a | | dosimetry |
| mixture | litter 0.035% of n-BTPT | n-BTPT state | (ppm) |
| LIT-14 | 3.50 g of LAB-14 | coating a particle | 25.00 |
| LIT-15 | 2.33 g of LAB-16 | of bentonite | 24.00 |
| LIT-16 | 1.75 g of LAB-15 | | 19.20 |
| LIT-17 | 0.35 g of pure n-BTPT | powder | 15.80 |
| (REF) | | | |

Observations:

The litter mixture LIT-16 has the best performance in terms of reduction in ammonia release. Therefore a content of 20% of n-BTPT on a bentonite support may be adequate, especially because the particulate support of LAB-14 to LAB-16 is made of fine particles of bentonite, thereby increasing the potential contact surface with urea.

TABLE 7

| | Mass of sample to be | | Urea/ |
|---|---|---|---|
| | added in 1 kg of absorptive | | Urease |
| Litter | substrate to obtain a | | dosimetry |
| mixture | litter 0.035% of n-BTPT | n-BTPT state | (ppm) |
| LIT-18 | 1.75 g of LAB-17 | coating a particle | 23.00 |
| | | of bentonite | 20.00 |
| LIT-19 | 0.35 g of pure n-BTPT | | 27.20 |
| (REF) | | | 15.00 |

Observations:

Each dosimetry test on LIT-18 and LIT-19 has been reproduced twice. The dosimetry results for coated particulate support (LIT-18) are more stable than for pure n-BTPT powder (LIT-19).

### Example 4

### n-BTPT on Bentonite Particulate Support

Further experiments have been performed by using bentonite (sieved at 40-100 mesh) as particulate support and n-BTPT as odor-retardant agent.

Prior to mixing with n-BTPT, the particles of bentonite were heated in an oven at 75° C. The heated particles of bentonite were then mixed with n-BTPT in a blender/mixer (placed in a warm water bath) such that n-BTPT melted by contact with the particles. The mixing step was also performed in a rotary evaporator. After cooling, precise quantities of the produced particles of anti-odor material were mixed with 1 kg of absorptive substrate (90% bentonite/10% limestone) so as to prepare litter mixture samples.

Dosimetry tests were then realised to evaluate the performance of the litter samples by measuring the ammonia release (in ppm) (Table 8).

TABLE 8

| | | Bentonite mass | | Urea/Urease |
|---|---|---|---|---|
| Litter | n-BTPT | in the rotary | n-BTPT | dosimetry |
| mixture | (%) | evaporator (g) | state | (ppm) |
| LIT-20 | 20% | 50 | coating a | 32.00 |
| LIT-21 | 10% | 50 | particle of | 29.40 |
| LIT-22 | | 100 | bentonite | 25 |
| LIT-23 | | 200 | | 31 |
| LIT-24 | 100% | — | powder | 20 |
| (REF) | | | | |

Observations:

The preparation of the anti-odor material with bentonite in the rotary evaporator provides homogeneous particles of anti-odor material. The dosimetry results of the n-BTPT coated particles and the powder of n-BTPT are quite similar.

### Example 5

### n-BTPT on Arcillite Support

Further experiments were performed using arcillite as particulate support. The arcillite was pre-heated to 70° C. and the powder of n-BTPT was poured onto the pre-heated support during mixing of the same, so as to melt n-BTPT and succeed a homogeneous coating of n-BTPT on the particulate support. The resulting anti-odor material was then cooled at room temperature to allow crystallization of the n-BTPT on the particulate support. Dosimetry tests were then realised to evaluate the performance of the litter samples by measuring the ammonia release (in ppm) (Table 9).

TABLE 9

| | Mass of sample to be | | Urea/ |
|---|---|---|---|
| | added in 1 kg of absorptive | | Urease |
| Litter | substrate to obtain a | | dosimetry |
| mixture | litter 0.035% of n-BTPT | n-BTPT state | (ppm) |
| LIT-31 | 1.17 g of a 30% n-BTPT | coating a particle of | 15 |
| | containing sample | arcillite | 19 |
| LIT-32 | 0.88 g of a 40% n-BTPT | coating a particle of | 15.8 |
| | containing sample | arcillite | 21.5 |
| LIT-33 | 0.35 g of pure n-BTPT | powder | 24 |
| (REF) | | | |

Observations:

Arcillite support seems to be better suited as the ammonia release reduction is higher than for other tested particulate supports.

### Example 6

Various particles of anti-odor material according to the present invention have been analyzed to evaluate the maximum content of n-BTPT that may be supported onto particulate supports of various nature, as provided in Table 10.

TABLE 10

| | Maximum quantity of |
|---|---|
| | n-BTPT on support in |
| Particulate support | one application (%) |
| Montmorillonite | 40 |
| Attapulgite | 40 |
| Cotton fabric | 50 |
| Litter particles of corn and wheat | 20 |
| Corn cob | 20 |
| Activated carbon | 30 |
| Granulated cellulose (Yesterday's News ®) | 20 |
| Paper sludge-based granules | 30 |

A n-BTPT charge between 30% and 40% on a montmorillonite particulate support is optionally chosen for the manufacture of the anti-odor material so as to respect the acceptable limits of n-BTPT quantities to be added in the litter.

### Example 7

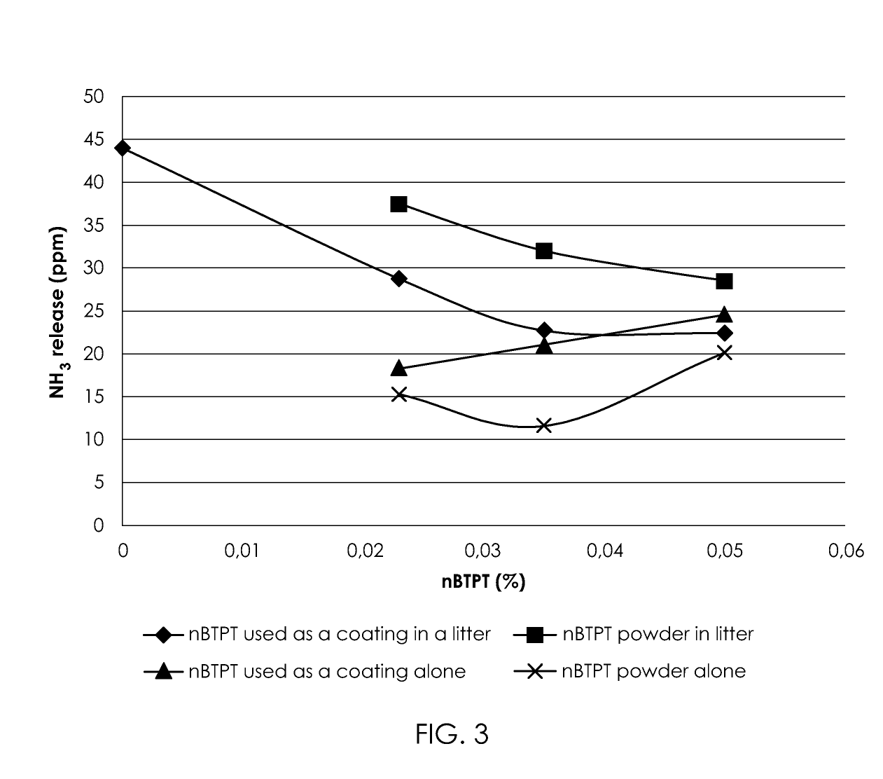

FIG. 3 is a graph of the ammonia release expressed in ppm as a function of n-BTPT concentration in the sample (in pure n-BTPT equivalent). Four series of samples were studied: a sample of anti-odor material including a particulate support coated with n-BTPT (series 1), a sample of n-BTPT powder (series 2), a sample of animal litter including an absorptive substrate mixed with the n-BTPT coated particulate support (series 3) and a sample of animal litter including the absorptive substrate mixed with powder of n-BTPT (series 4); each series including various n-BTPT concentrations. The absorptive substrate was a mixture of 80% bentonite and 20% limestone. The particulate support was particles of montmorillonite. Dosimetry tests were performed after contacting samples of series 3 and series 4 with a solution of urea prepared as above-mentioned. Dosimetry tests were performed on series 1 and series 2 without contacting the samples with a solution of urea. The dosimetry results are gathered in following Tables 11 and 12 and were used to draw the graph of FIG. 3.

TABLE 11

| | | | | Mass to | | |
|---|---|---|---|---|---|---|
| | | | | be added | | |
| | | | | in 1 kg of | Final pure n- | |
| | n-BTPT in | particulate | | absorptive | BTPT | Urea/Urease |
| | the anti-odor | support | n-BTPT | substrate | equivalent | dosimetry |
| | material (%) | (%) | (g) | (g) | (%) | (ppm) |
| REF | 0 | | | 0 | 0 | 44 |
| n-BTPT | 30 | 70 | 236 | 0.767 | 0.023 | 29 |
| coated on | | | | 1.167 | 0.035 | 23 |
| support | | | | 1.667 | 0.050 | 23 |
| (series 3) | | | | | | |

| | | | | | |
|---|---|---|---|---|---|
| n-BTPT | 100 | — | 0.230 | 0.023 | 38 |
| powder | 100 | | 0.350 | 0.035 | 32 |
| (series 4) | 100 | | 0.500 | 0.050 | 29 |

TABLE 12

| | | | | | Final pure n- | |
|---|---|---|---|---|---|---|
| | n-BTPT in the | particulate | | Mass to | BTPT | Urea/Urease |
| | anti-odor | support | n-BTPT | be | equivalent | dosimetry |
| | material (%) | (%) | (g) | analyzed | (%) | (ppm) |
| n-BTPT | 30 | 70 | 236 | 0.307 | 0.023 | 18 |
| coated on | | | | 0.467 | 0.035 | 21 |
| support | | | | 0.667 | 0.050 | 25 |
| (series 1) | | | | | | |

| | | | | | |
|---|---|---|---|---|---|
| n-BTPT | 100 | — | 0.092 | 0.023 | 15 |
| powder | | | 0.140 | 0.035 | 12 |
| (series 2) | | | 0.200 | 0.050 | 20 |

The animal litter (REF) including 0% of n-BTPT, i.e. without odor control, naturally releases 45 ppm of ammonia in contact with the urea sample. For 0.023% n-BTPT concentration, the powder of n-BTPT (series 2) releases 15 ppm of ammonia and the montmorillonite coated with 0.023% n-BTPT (series 1) releases 18 ppm. Therefore, the use of n-BTPT enables to reduce the ammonia release and the reduction in ammonia release from the montmorillonite coated with n-BTPT is not as high as from n-BTPT in powder form.

Additionally, the graph shows that at higher n-BTPT concentrations, the n-BTPT coated support or the n-BTPT powder may release more ammonia than the litter itself. Indeed, it has been found that n-BTPT naturally contains or releases ammonia (i.e. ammonia which does not originate from the hydrolysis of urea) (see dosimetry results from Table 12). Therefore, the initial ammonia amount which may be naturally released from the n-BTPT has to be subtracted to estimate the real amount of ammonia coming from the hydrolysis of urea.

For a 0.023% n-BTPT concentration, the powder of n-BTPT (series 2) releases 15 ppm of ammonia, whereas the urea-impregnated mixture of n-BTPT powder and litter (series 4) releases 38 ppm. Therefore, the ammonia release due to the hydrolysis of urea is 23 ppm (38 ppm-15 ppm). As the 0% n-BTPT litter (REF) releases 45 ppm of ammonia after 48 hours, the 0.023% n-BTPT containing mixture of n-BTPT powder and litter reduces the ammonia release by 49% [(23/45)×100]. The same mixture reaches 100% efficiency in ammonia release reduction at around 0.04% n-BTPT concentration (as may be seen by the crossing point of the two curves corresponding to the ammonia release of the particulate support coated with n-BTPT and of the mixture of litter and particulate support coated with n-BTPT).

### CONCLUSION

The true reduction in ammonia release due to the action of urease (catalyst) on the hydrolysis of urea can vary from 0 to 100% depending on the n-BTPT concentration contained in the litter. Therefore, an optimal concentration of n-BTPT may preferably be used in the litter and having a n-BTPT concentration higher than this optimal value may lead to an increase in the ammonia release.

1. A process for producing an anti-odor material comprising:
providing an absorptive substrate;
mixing a crystallized odor-retardant agent with the absorptive substrate for producing the anti-odor material, the crystallized odor retardant agent comprising a phoshorotriamide of formula R₁R₂R₃N₃PR₄, wherein R₁, R₂ and R₃ are hydrogen atoms or alkyl groups, and R₄ is an oxygen or a sulfur atom.

2.-6. (canceled)

7. A process for producing an anti-odor material for use in animal litter, the process comprising:
providing an absorptive substrate; and
mixing an odor-retardant agent in a melted phase with the absorptive substrate for producing the anti-odor material.

8. The process of claim 7, comprising melting the odor-retardant agent in a powder form before mixing with the absorptive substrate, so as to obtain the odor-retardant agent in the melted phase.

9. The process of claim 8, wherein the melting is performed by heating the odor-retardant agent in powder form at a heating temperature between 50° C. and 80° C.

10. (canceled)

11. (canceled)

12. The process of claim 8, wherein the melting is performed by heating the odor-retardant agent in the powder form to at least an odor-retardant agent melting point Tₘ to form the odor-retardant agent in the melted phase.

13. The process of claim 7, wherein the mixing is performed during a mixing time which is sufficient to coat a surface of the absorptive substrate with a layer of the odor-retardant agent in the melted phase.

14. The process of claim 12, further comprising cooling the anti-odor material to a temperature below the odor-retardant agent melting point Tₘ to transform the melted phase of the odor-retardant agent into a crystallized phase.

15.-44. (canceled)

45. An animal litter comprising:
an absorptive substrate; and
an anti-odor material comprising:
a crystallized odor-retardant agent comprising a phosphorotriamide of formula: R₁R₂R₃N₃PR₄, wherein R₁, R₂ and R₃ are hydrogen atoms or alkyl groups, and R₄ is an oxygen or a sulfur atom.

46. The animal litter of claim 45, wherein the absorptive material comprises a non-clumping clay-based compound, a clumping clay-base compound, a limestone-based compound, a silica-based compound, a cellulose-based compound, a cellulose derivatives-based compound, an agricultural waste-based compound, a soil-based compound or a combination thereof.

47. The animal litter of claim 49, wherein the particulate support is made with the same material as the absorptive substrate.

48. The animal litter of claim 50, wherein the mass percentage of absorptive substrate with respect to the anti-odor material is between 5 wt % and 40 wt % so as to obtain an equivalent pure n-BTPT content between 0.005 wt % and 0.05 wt %.

49. The animal litter of claim 45, wherein the anti-odor material further comprises a particulate support, the odor retardant agent being associated with the particulate support so as to coat a surface of the particulate support with a layer of odor-retardant agent.

50. The animal litter of claim 45, wherein the crystallized odor retardant agent results from the transition from a melted phase into the crystallized phase.

51. The animal litter of claim 45, wherein the crystallized odor-retardant agent is crystallized N-(n-butyl)thiophosphoric triamide (n-BTPT), having the molecular formula C₄H₁₄N₃PS with the following structure:

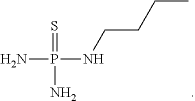

52. The animal litter of claim 45, wherein the particulate support comprises a plurality of particles having a mesh size between 20 and 100.

53. The animal litter of claim 46, wherein the clumping clay-based compound and/or the non-clumping clay-based compound comprise bentonite, montmorillonite, arcillite, attapulgite or a combination thereof.

54. The process of claim 1, wherein the crystallized odor-retardant agent results from the transition from a melted phase into a crystallized phase.

55. The process of claim 1, wherein the crystallized odor-retardant agent is crystallized N-(n-butyl)thiophosphoric triamide (n-BTPT), having the molecular formula C₄H₁₄N₃PS with the following structure:

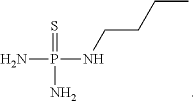

56. The process of claim 1, further comprising coating the crystallized odor-retardant agent on a particulate support prior to mixing the crystallized odor-retardant agent with the absorptive substrate.

57. The process of claim 1, wherein the absorptive substrate comprises a non-clumping clay-based compound, a clumping clay-base compound, a limestone-based compound, a silica-based compound, a cellulose-based compound, a cellulose derivatives-based compound, an agricultural waste-based compound, a soil-based compound or a combination thereof.
